(19) United States
(12) Patent Application Publication
(10) Pub. No.: US 2022/0174941 A1
(43) Pub. Date: Jun. 9, 2022

(54) Method of Increasing Bone Cell Viability

(71) Applicant: Seaspine, Inc., Carlsbad, CA (US)

(72) Inventors: Frank Vizesi, Carlsbad, CA (US); Jiawei He, Carlsbad, CA (US); Jeremy Lombardo, Carlsbad, CA (US)

(21) Appl. No.: 17/544,229

(22) Filed: Dec. 7, 2021

Related U.S. Application Data
(60) Provisional application No. 63/122,847, filed on Dec. 8, 2020.

(51) Int. Cl. A01N 1/02; A61L 27/36; C12N 5/077

(52) U.S. Cl. CPC A01N 1/0226; A61L 27/3608; C12N 5/0669; C12N 5/0654; A61L 27/3683

(57) ABSTRACT

A method of maintaining cellular viability of harvested bone, where the method includes: providing a source of bone or bone particles; combining the bone or the bone particles with a sterile solution; and storing the bone or the bone particles in the sterile solution until their introduction into a patient.

### CROSS-REFERENCE TO RELATED APPLICATIONS

This application claims the benefit of priority to U.S. Provisional Application No. 63/122,847, filed Dec. 8, 2020, the contents of which are incorporated by reference in their entirety.

### BACKGROUND

Implants for fusion procedures, spinal or otherwise, may promote bone growth that fuses adjacent bone (e.g. vertebrae in the case of spinal fusions) together. An implant may promote fusion by a number of mechanisms including osteogenesis, osteoinduction, and/or osteoconduction. Osteogenesis refers to the formation of new bone by cells contained within the implant. Osteoinduction refers to a chemical process by which osteogenesis may be induced, where molecules within the implant are converted to material used by the patient to form bone. Osteoinduction is regularly observed during the bone healing process. Osteoinduction typically includes recruitment of immature cells and the stimulation of these cells. For example, where there is a fracture, the bone healing may be primarily dependent on osteoinduction. Osteoconduction refers to a process where a matrix of the implant forms a scaffold on which cells are able to form new bone. The success of overall bone grafting or bone formation may be attributable to the relative success of the osteoinduction, osteoconduction, and/or osteogenesis processes. The three-dimensional properties of the bone or bone portion utilized in the graft may influence the osteoconductive properties.

In an autograft procedure, for example, bone may be harvested from the patient and is then typically stored, for example in a surgical basin, until it is needed for the fusion or other procedure. The time duration of storage may range from being used immediately, to upwards of 6 hours in some extensive surgeries. In a typical orthopedic or spine procedure, it may be, for example, 15 minutes to 4 hours. However, little is known about the effect of the cellular component of the harvested bone on the success of the subsequent implantation for fusion. The cellular component of the bone may include osteoblasts, osteocytes, osteoclasts, and osteogenic (or stem) cells. Osteoblasts may synthesize the uncalcified extracellular matrix called the osteoid, which may subsequently become calcified to form bone. As the osteoid mineralizes, osteoblast cells may be disposed in the lamellae in lacunae, where they mature into osteocytes. The osteocytes may regulate bone mass. Osteoclasts are large, multinucleated cells that function to resorb bone by releasing H+ ions and lysosomal enzymes.

### SUMMARY

In one aspect, a method of maintaining cellular viability of harvested bone is disclosed herein, the method comprising: providing a source of bone or bone particles; combining the bone or bone particles with a sterile solution; storing the bone or bone particles in the sterile solution until introduction into a patient.

In some embodiments, providing a source of bone or bone particles comprises harvesting bone from the patient.

In some embodiments, the sterile solution comprises a salt solution, including a saline solution. In other embodiments, the sterile solution includes one or more of saline or other salt solution, a buffer, a preservative, an excipient, a gelling agent, a nutrient, an electrolyte, a carrier, a temperature-sensitive polymer, a solubilizing agent, a stabilizing agent, a tonicity modifier, a bulking agent, a viscosity enhancer or reducer, a surfactant, or any combination or mixture thereof.

In some embodiments, the combining step further includes wetting the bone or bone particles with the sterile solution. In some other embodiments, the bone or bone particles comprise cortical bone, cancellous bone, or both cortical and cancellous bone. In one or more embodiments, the bone or bone particles may also include the associated bone marrow, in whole or in part. In one or more embodiments, the bone marrow may be used alone, or the bone marrow may be used with the bone and/or bone particles.

In another aspect, a method of extending cellular viability of harvested bone for an autograft is disclosed herein, the method comprising: providing a source of bone or bone particles; combining the bone or bone particles with a sterile solution; storing the bone or bone particles in the sterile solution until autograft introduction into a patient.

In some embodiments, providing a source of bone or bone particles comprises harvesting bone from the patient.

In some embodiments, the sterile solution includes saline. In other embodiments, the sterile solution includes one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

In some embodiments, the combining step further includes wetting the bone or bone particles with the sterile solution. In some embodiments, the bone or bone particles comprise cortical bone, cancellous bone, or both cortical and cancellous bone.

In yet another aspect, a surgical method for instilling harvested bone or bone particles into a patient is disclosed herein, the method including: obtaining a bone or bone particles from the patient; combining the bone or bone particles with a sterile solution; storing the bone or bone particles in the sterile solution; removing the bone or bone particles from the sterile solution; and introducing the bone or bone particles into the patient.

In some embodiments, the combining step further includes wetting the bone or bone particles with the sterile solution.

In some embodiments, the sterile solution comprises saline. In some other embodiments, the sterile solution includes one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

In some embodiments, the bone or bone particles comprise cortical bone, cancellous bone, or both cortical and cancellous bone.

In still another aspect, a kit for maintaining cellular viability of harvested bone or bone particles for an autograft is disclosed herein, the kit including: a vessel for holding said harvested bone or bone particles; and a vessel for holding a sterile solution.

In some embodiments, the sterile solution comprises saline. In some other embodiments, the sterile solution comprises one or more of saline, a buffer, a preservative, an excipient, a gelling agent, a nutrient, an electrolyte, a carrier, a temperature-sensitive polymer, a solubilizing agent, a stabilizing agent, a tonicity modifier, a bulking agent, a viscosity enhancer or reducer, a surfactant, or any mixture thereof.

In some embodiments, the bone or bone particles comprise cortical bone, cancellous bone, or both cortical and cancellous bone.

In some embodiments, the harvested bone or bone particles are contained in a first vessel, and the sterile solution is contained in a second vessel. In other embodiments, the harvested bone or bone particles and the sterile solution are combined in a single vessel.

In still yet another aspect, a kit for extending cellular viability of harvested bone or bone particles for an autograft is disclosed herein, the kit includes: a vessel for holding the harvested bone or bone particles; and a vessel for holding a sterile solution.

In some embodiments, the sterile solution comprises saline. In some other embodiments, the sterile solution comprises one or more of saline, a buffer, a preservative, an excipient, a gelling agent, a nutrient, an electrolyte, a carrier, a temperature-sensitive polymer, a solubilizing agent, a stabilizing agent, a tonicity modifier, a bulking agent, a viscosity enhancer or reducer, a surfactant, or any mixture thereof.

In some embodiments, the bone or bone particles comprise cortical bone, cancellous bone, or both cortical and cancellous bone. In some embodiments, the harvested bone or bone particles are contained in a first vessel, and the sterile solution is contained in a second vessel. In other embodiments, the harvested bone or bone particles and said sterile solution are combined in a single vessel. In still other embodiments, the kit further includes instructions for mixing the bone or bone particles with the sterile solution.

In one or more embodiments, the sterile solution may be implanted with the bone or bone particles and may help form the bone particles into a cohesive mass.

### BRIEF DESCRIPTION OF THE FIGURES

FIGS. 1A-B are histological cross-sections of a fusion site where a viable iliac crest sample was grafted as described in Example 1. FIG. 1A is a broad view of the site; FIG. 1B is a zoomed in view of a portion of FIG. 1A.

FIGS. 2A and 2B are histological cross-sections of the fusion site where partially devitalized iliac crest was grafted as described in Example 1. FIG. 2B is a zoomed in view of a portion of FIG. 2A.

FIGS. 3A and 3B are histological cross-sections of a fusion site where a devitalized iliac crest sample was grafted as described in Example 1. FIG. 3A is a broad view of the site;

FIG. 3B is a zoomed in view of a portion of FIG. 3A.

FIGS. 4A, 4B, and 4C are representative μCTs from each group as described in Example 1. FIG. 4A is the μCT of the viable iliac crest sample; FIG. 4B is the μCT of the partially devitalized iliac crest sample; FIG. 4C is the μCT of the devitalized iliac crest sample.

FIGS. 5A and 5B are histological cross-sections of the fusion site where viable iliac crest (extended anesthesia control) was grafted as described in Example 2, where FIG. 5B is a zoomed in view of a portion of FIG. 5A.

FIGS. 6A and 6B are histological cross-sections of the fusion site where devitalized iliac crest (air dried) was grafted as described in Example 2, where FIG. 6B is a zoomed in view of a portion of FIG. 6A.

FIGS. 7A and 7B are histological cross-sections of the fusion site where hydrated iliac crest (immersed in saline) was grafted as described in Example 2, where FIG. 7B is a zoomed in view of a portion of FIG. 7A.

FIGS. 8A, 8B, and 8C are representative μCTs from each group as described in Example 2. FIG. 8A is the μCT of the viable iliac crest (extended anesthesia control) sample;

FIG. 8B is the μCT of the devitalized iliac crest (air dried) sample; FIG. 8C is the μCT of the hydrated iliac crest (immersed in saline).

FIG. 9 is a graph illustrating in vitro bone cell viability results for “dry” or control samples of Example 2.

FIGS. 10A and 10B are graphs illustrating results of the bone viability experiment described in Example 2. FIG. 10A illustrates in vitro bone cell viability results for “dry” or control samples of Example 2; FIG. 10B illustrates in vitro bone cell viability results for “wet” or treatment samples of Example 2.

FIG. 11 is a graph illustrating the results of a bone cell viability experiment using an alginate gel.

FIG. 12 is a graph illustrating the results of a bone cell viability experiment using a carboxymethyl cellulose gel.

### DETAILED DESCRIPTION

The standard technique employed in spinal fusion is traditionally an autograft. The surgeon may harvest bone from one part of the patient's body and graft the harvested bone to another part of the body, for example the spine. Bone is typically harvested from an area of the body where its removal will not be problematic for the patient. In the example of spinal fusions, the bone may be harvested from the iliac crest or another local bone. As mentioned previously, little is known about the effect of the cellular component of the harvested bone on the success of the subsequent implantation for fusion. The effect of cellular viability on fusion procedures was investigated. As described in Example 1 herein, it is clear that the cellular component of the harvested bone is important to the success of the fusion.

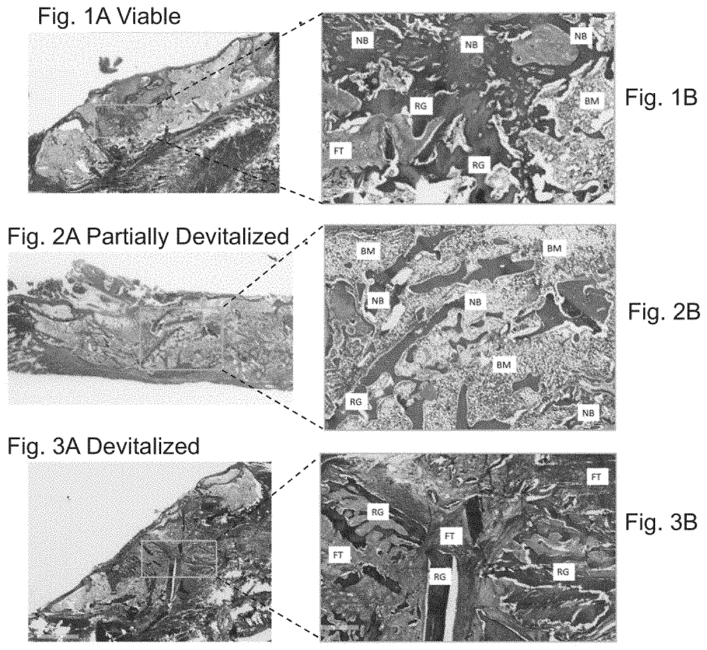

The bone placed in a sterile vessel (e.g. a surgical basin) may be stored, in some instances, for as long as 8 hours, though depending on the complexity of the surgical procedure. For example, when a particle of bone is collected at the beginning of the procedure and then left unused until the end of the procedure, this period may be even longer than 8 hours. Given the importance of the cellular component of the harvested bone, described in Example 1 and discussed herein and illustrated in FIGS. 1A-B and 2A-B, methods of maintaining or extending cellular viability of the harvested bone may be desired. These methods may generally begin with providing a bone. In some instances, this bone may be harvested from the same patient in which the bone is being implanted (e.g. an autograft). In other instances, this bone may be from another source (e.g. an allograft). The bone may, in some instances, be cortical bone, cancellous bone, or a combination thereof. Regardless of the source, the bone may take a variety of forms, for example, the bone may be morselized into chips, ground into a powder, reduced to bone fibers of various lengths, and the like. prior to implantation. In contrast to conventional methods, where the bone is placed into a surgical basin and remains there unaltered until implantation, the bone may be combined with a sterile solution.

In some instances, the sterile solution may be added to the surgical basin in a sufficient quantity so as to fully submerge the bone until the appropriate time to implant the bone into the patient. In other instances, the sterile solution may be sprayed, poured, or otherwise added to the bone at predetermined time intervals until the appropriate time to implant the bone. For example, the sterile solution may be sprayed, poured on, and the like, every 2 minutes, 5 minutes, 10 minutes, or any other interval sufficient for the surface of the bone to be wet. The wetting time may also be adjusted to maintain the bone in a wetted state so that the bone does not become dry. In some instances, the sterile solution may be saline. In other instances, the sterile solution may be saline, a buffer, a preservative (including a cryoprotectant), an excipient, a gelling agent, a nutrient, an electrolyte, a carrier, a temperature-sensitive polymer, a solubilizing agent, a stabilizing agent, a tonicity modifier, a bulking agent, a viscosity enhancer or reducer, or a surfactant, either alone or in any combination. Example preservation solutions that may be used include, for example, University of Wisconsin (UW) solution and Collins solution. Other relevant preservation solutions, including for example glucose with electrolytes and/or Plasmalyte A, may also be employed in accordance with the current invention.

In some instances, it may be desirable to include a growth medium, either alone or in combination with one or more sterile solution(s) as described herein.

The appropriate solution (buffers, and the like) may be mechanically agitated in two (2) or three (3) dimensions using, for example, a shaking apparatus, and/or cycled through the vessel using a pumping mechanism. The mechanism of action is improved transport of the solution to the graft bone material through these mechanical means.

In instances where the bone is submerged in the sterile solution (regardless of the composition of the solution), the bone may be removed from the sterile solution once the surgeon has exposed the desired implantation site and is ready to implant or place the bone into the patient. The bone may be used as is, or may be rinsed to remove the sterile solution prior to implantation. The bone may then be introduced into the patient. In some instances, this introduction may be in combination with another biologic material at the surgeon's discretion to extend the bone graft (e.g., an autograft) or in some combination with other biological materials prior to implantation. In one embodiment, the sterile solution comprises a gel-like substance which helps to form the bone chips into a cohesive putty to aid in implantation and to provide nutrients after implementation.

In some instances, a kit may be used to facilitate the maintenance and/or extension of cellular viability of a harvested bone for an autograft. Such a kit may include a vessel for holding the harvested bone. In some instances, this vessel may be a surgical basin or the like. In other instances, this vessel may be a container capable of being closed, for example with a lid. The kit may also include a vessel for holding the sterile solution.

In some instances, the kit may include two separate vessels, for example a surgical basin for receiving the harvested bone and a sealed vessel containing the sterile solution included therein. After harvesting the bone and placing it in the provided surgical basin the surgeon may then add the sterile solution provided in the kit.

In other instances, the kit may include only a single vessel for holding both the sterile solution and the harvested bone. As a non-limiting example, the kit may include a surgical basin or the like that contains the sterile solution in advance of receiving the harvested bone.

### EXAMPLES

### Example 1

The fusion rate of viable bone, devitalized bone, and partially devitalized bone recovered from the iliac crest of rabbits was compared in an effort to evaluate the contribution of the cellular fraction of an autograft on new bone growth in a spinal fusion. Devitalized bone samples were rinsed twice with sterile saline and put through a freeze/thaw cycle to inactivate the cells. The bone samples were frozen by placing the subject bone into a sterile container, then submerging the container with the bone in dry ice. By freezing the bone using this method or a comparable freezing method, the bone cells die due to the formation of ice crystals. This method provides a relatively simple and fast way to kill the bone cells. Partially devitalized bone samples were vigorously rinsed twice with sterile saline to remove nonadherent cell populations.

The viable, devitalized, and partially devitalized bones were grafted into posterolateral spine per the standard Boden rabbit spinal fusion model; the grafts were placed into the left and right sides of the spine. The results of this experiment are described in Table 1.

TABLE 1

| | # of | | |
|---|---|---|---|
| Grafted Material | Rabbits | Fusion Rate | |
| Viable iliac crest autograft | 8 | 7/12 | (58%)*** |
| Partially devitalized iliac crest autograft | 8 | 12/14 | (86%)*** |
| Devitalized iliac crest autograft | 8 | 0/16 | (0%) |

***denotes statistical significance vs. devitalized group at p < 0.001

Two of the animals that received the viable iliac crest graft and one of the animals that received the partially devitalized iliac crest graft died due to post-operative complications and were not replaced. FIGS. 1A-B are histological cross-sections of the fusion site where viable iliac crest was grafted, where FIG. 1B is a zoomed in view of a portion of FIG. 1A. As is clear in FIGS. 1A-B little residual graft (RG) remains, while there are numerous areas of new bone (NB) formation. FIG. 1B further illustrated substantial amounts of bone marrow (BM) with limited fibrous tissue (FT) visible in the cross-section. Notably, BM is an indicator of the presence of more mature bone. FIGS. 2A-B are histological cross-sections of the fusion site where partially devitalized iliac crest was grafted, where FIG. 2B is a zoomed in view of a portion of FIG. 2A. As is clear in FIGS. 2A-B little residual graft (RG) remains, while there are numerous areas of new bone (NB) formation. FIG. 2B further illustrated substantial amounts of bone marrow (BM) with limited fibrous tissue (FT) visible in the cross-section. FIGS. 3A-B are histological cross-sections of the fusion site where devitalized iliac crest was grafted, where FIG. 3B is a zoomed in view of a portion of FIG. 3A. The devitalized iliac crest autograft, as illustrated in FIGS. 3A-B, had significantly more residual graft (RG) remaining and no areas of new bone formation or bone marrow (BM) throughout the fusion mass. FIG. 3B also illustrates significant fibrous tissue (FB) visible in the cross-section. As is clear from the Figures, the viability of the grafted bone significantly impacted the success of the fusion, thus indicating the importance of the cellular component on the success of the fusion.

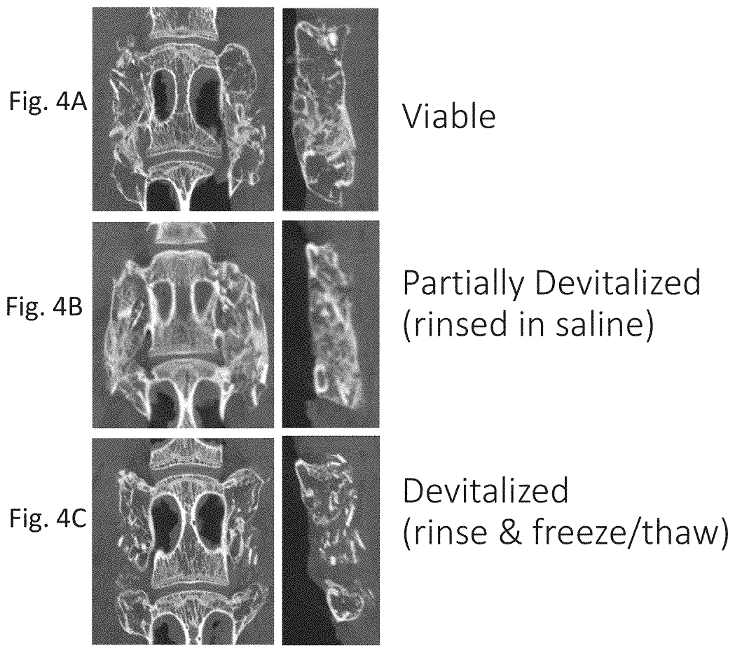

FIGS. 4A-C are representative μCTs from each group, where FIG. 4A is the μCT of the viable iliac crest sample, FIG. 4B is the μCT of the partially devitalized iliac crest sample, and FIG. 4C is the μCT of the devitalized iliac crest sample. The μCT reconstructions of the viable and partially devitalized groups (FIG. 4A-B) displayed bilateral fusion with contiguous bone masses bridging between the L4-L5 transverse processes (TPs). In contrast, the devitalized autograft group (FIG. 4C) lacked bone bridging between the TPs and, instead, disparate islands of bone are noted between the TPs.

### Example 2

Twenty-four female rabbits (Western Oregon Rabbit Company, Philomath, OR) approximately 6 months old were used following ethical approval. Harvested and morselized autograft was either dried in air for 90 mins or immersed in saline for 90 min prior to implantation. A control arm was included where each animal in this group was maintained under anesthesia for the same total amount of time as the other groups, in case of any negative effects of an extended anesthesia regimen. For autograft harvest, a midline skin incision over the caudal lumbosacral spine was utilized to approach the L4-5 interspaces and iliac crests. Autograft from the iliac crests (2.5-3 cc per side) was harvested and morselized to pieces 1-4 mm in size. For the air dried autograft condition, the morselized autograft was left out to dry for 90 min under ambient conditions and then loaded into syringes for deployment. For the saline autograft condition, the morselized autograft was fully immersed in saline for 90 min and then the graft material was loaded into syringes for deployment. Morselized autograft were grafted into posterolateral spine per the standard Boden rabbit spinal fusion model; the grafts were placed into the left and right sides of the spine. The results of this experiment are described in Table 2.

TABLE 2

| | # of | | |
|---|---|---|---|
| Grafted Material | Rabbits | Fusion Rate | |
| Viable iliac crest autograft (extended | 8 | 11/16 | (69%)*** |
| anesthesia) | | | |
| Devitalized iliac crest autograft (air dried) | 8 | 0/16 | (0%) |
| Hydrated iliac crest autograft (immersed | 8 | 14/16 | (88%)*** |
| in saline) | | | |

***denotes statistical significance vs. devitalized group at p < 0.001

Fusion rate was statistically significantly lower for the devitalized autograft group (0% fusion) compared to the viable iliac crest autograft group (69% fusion), suggesting that leaving bone out to dry on the back table negatively affects fusion performance. Fusion rates were statistically similar for the viable iliac crest and hydrated iliac crest group (69% vs. 88% fusion), suggesting that immersion in saline helps to protect and maintain fusion performance of autograft.

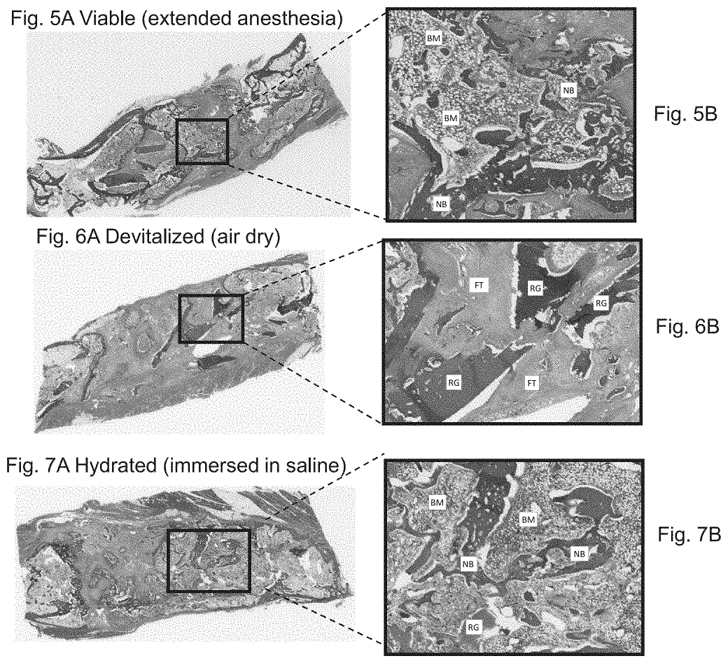

FIGS. 5A-5B are histological cross-sections of the fusion site where viable iliac crest (extended anesthesia control) was grafted, where FIG. 5B is a zoomed in view of a portion of FIG. 5A. As is clear in FIGS. 5A-B little residual graft (RG) remains, while there are numerous areas of new bone (NB) formation and bone marrow (BM). FIG. 5B further illustrates substantial amounts of bone marrow (BM) with limited fibrous tissue (FT) visible in the cross-section. FIGS. 6A-B are histological cross-sections of the fusion site where devitalized iliac crest (air dried) was grafted, where FIG. 6B is a zoomed in view of a portion of FIG. 6A. As is clear in FIGS. 6A-B substantially more residual graft (RG) remains, while there are few areas of new bone (NB) formation or bone marrow (BM). FIG. 6B further illustrated substantial amounts of fibrous tissue (FT) visible in the cross-section. FIGS. 7A-B are histological cross-sections of the fusion site where hydrated iliac crest (immersed in saline) was grafted, where FIG. 7B is a zoomed in view of a portion of FIG. 7A. The hydrated iliac crest autograft, as illustrated in FIGS. 7A-B, little residual graft (RG) remains, while there are numerous areas of new bone (NB) formation and bone marrow (BM). FIG. 7B also illustrates substantial amounts of bone marrow (BM) visible in the cross-section. As is clear from the Figures, the viability of the grafted bone significantly impacted the success of the fusion, thus indicating the importance of the cellular component on the success of the fusion.

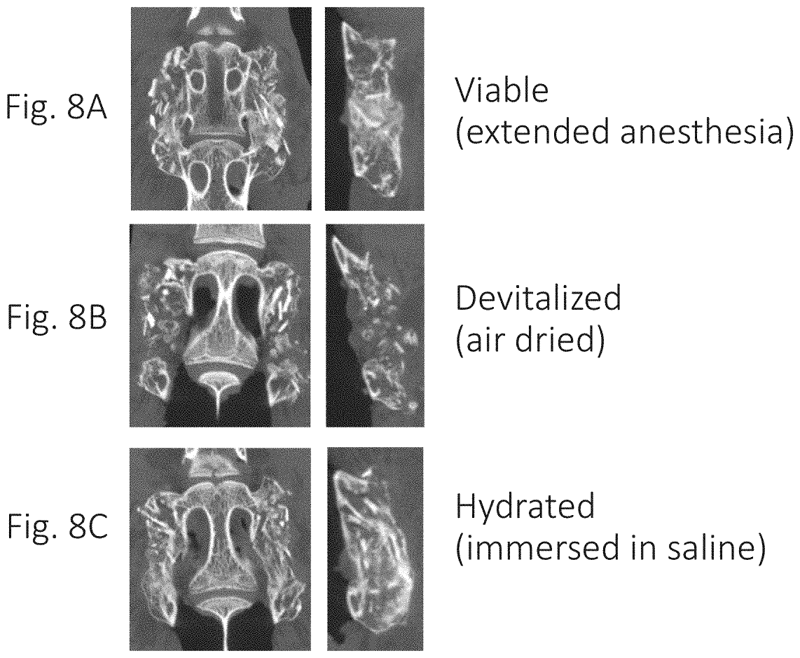

FIGS. 8A-8C are representative μCTs from each group, where FIG. 8A is the μCT of the viable iliac crest (i.e. the extended anesthesia control) sample, FIG. 8B is the μCT of the devitalized iliac crest (the air dried) sample, and FIG. 8C is the μCT of the hydrated iliac crest (the immersed in saline) sample. μCT reconstructions of the viable (extended anesthesia control) and hydrated (immersed in saline) groups (FIGS. 8A, 8C, respectively) displayed bilateral fusion with contiguous bone masses bridging between the L4-L5 transverse processes (TPs). In contrast, μCT reconstructions of the devitalized (air dried) autograft group (FIG. 8B) show a lack of bone bridging between the TPs and, instead, disparate islands of bone are noted between the TPs.

### Example 3

Femurs from two to three year old sheep were obtained, cut into 8 smaller pieces, and rinsed 3 times using saline at 37° C. with vigorous shaking to remove non-adherent cells. Washed bone was then morselized into cancellous chips of bone varying from about 1 to about 4 millimeters in size. The chips were divided into two groups—a treatment group that remained immersed in saline and a control group left open to the air. Each sample contained 2 g of chips, and a minimum of 3 biological replicates were used per condition. Both groups were then left in ambient conditions in a laminar flow hood, simulating being left on a back table during a surgical procedure. Cell viability was determined through an alamarBlue assay (BioRad®) at 0, 0.5, 1, 2, 4, and 19 hours. AlamarBlue was diluted to a 1× working solution in cell culture media, per manufacturer's instructions, and chips were incubated for 30 minutes at 37° C. with shaking. Following incubation, 100 μL aliquots of the solution were transferred to a 96 well plate and the level of fluorescence produced was measured (535 nm excitation, 595 nm emission) using an Infinte 200 PRO plate reader (Tecan®). The alamarBlue assay quantifies cell viability by using a cell permeable and fluorescent indicator dye called resazurin. This intensity of fluorescence produced is proportional to the number of living cells respiring.

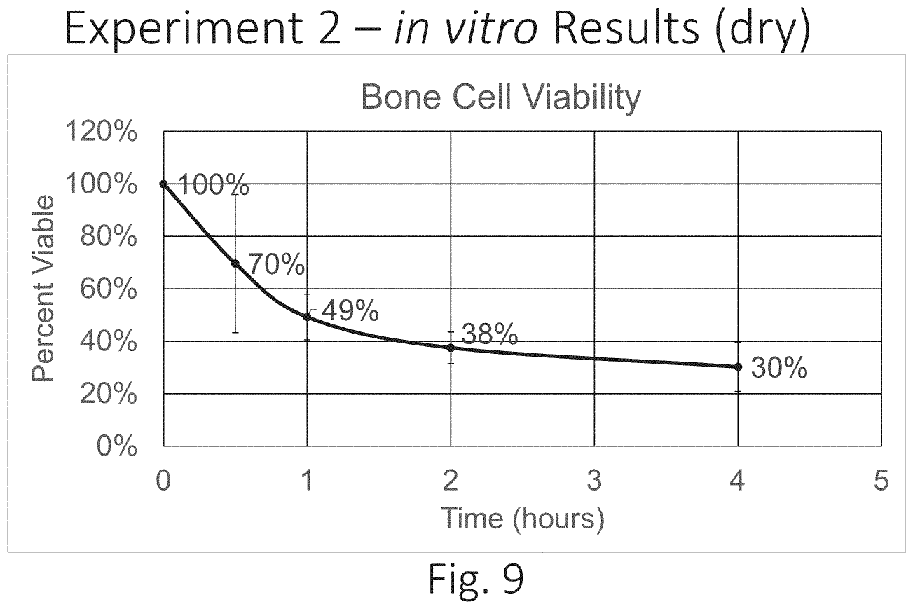

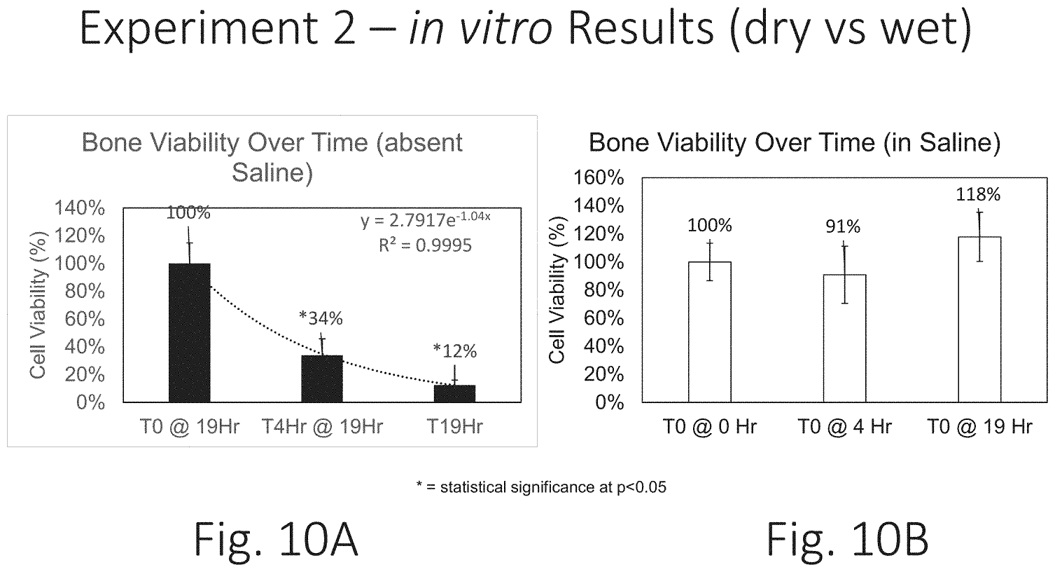

The results of the experiment are illustrated in FIGS. 9 and 10A-B. Fluorescent intensity results were normalized relative to the 0 hour condition in each figure. FIG. 9 illustrates the relatively rapid decline in bone cell viability over four hours in the control group that was not immersed in saline. As illustrated in FIG. 9, by hour four, the percent of viable cells had dropped to 30%. FIGS. 10A-B each illustrate the difference in bone cell viability over 19 hours between the control group that was not immersed in saline (FIG. 10A) and the treatment group that was immersed in saline (FIG. 10B). As indicated by the asterisk in FIG. 10A, the difference in cellular viability between the “dry” bone and the bone immersed in saline is statistically significant.

### Example 4

Femurs from cows approximately two to three years old were obtained, cut into smaller pieces, and morselized into cancellous chips of bone of about 4 mm in size. The chips were divided into three groups—one treatment group that remained immersed in saline, another treatment group that remained immersed in a gel, and a control group that was control group left open to the air. Two different gel formulations were tested, one consisted of carboxymethyl cellulose (CMC) dissolved in a saline solution and another consisted of alginate dissolved in a saline solution. All groups were then left in ambient conditions on a benchtop, simulating being left on a back table during a surgical procedure. Each sample contained 2 g of chips, and a minimum of 3 biological replicates were used per condition. Prior to cell viability assessment, samples were rinsed 2 times using saline at 37° C. with vigorous shaking to remove non-adherent cells. Cell viability was then determined through an alamarBlue assay (BioRad®) at 0, 2, and 4 hours. AlamarBlue was diluted to a 1× working solution in cell culture media, per manufacturer's instructions, and chips were incubated for 2 hours at 37° C. Following incubation, 100 μL aliquots of the solution were transferred to a 96 well plate and the level of fluorescence produced was measured (535 nm excitation, 595 nm emission) using an Infinite 200 PRO plate reader (Tecan®). The alamarBlue assay quantifies cell viability by using a cell permeable and fluorescent indicator dye called resazurin. This intensity of fluorescence produced is proportional to the number of living cells respiring.

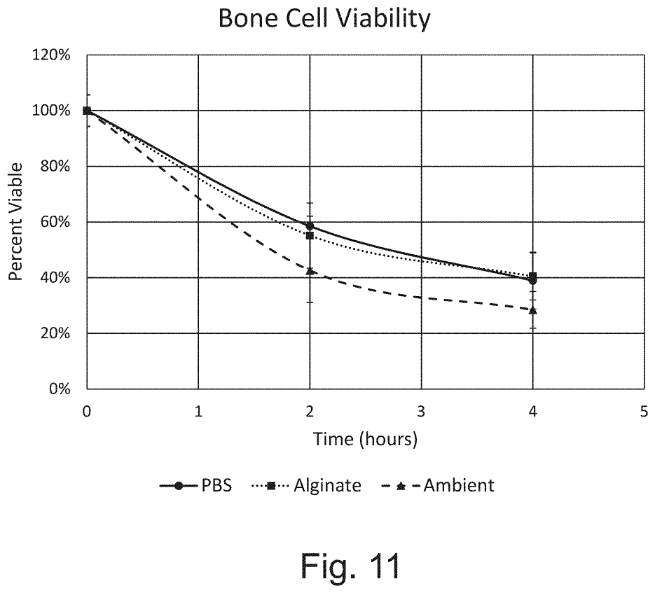

The results of an experiment using an alginate gel are illustrated in FIG. 11. Fluorescent intensity results were normalized relative to the 0 hour condition in each figure. FIG. 11 illustrates the relatively rapid decline in bone cell viability over four hours in the control group that was not immersed in saline or alginate gel. Bone cell viability in saline or alginate gel groups also declined with time, although less drastically relative to the control group. As illustrated in FIG. 11, by hour four, the percent of viable cells had dropped to 28% for the control group and 39% and 40% for the saline and alginate gel groups, respectively.

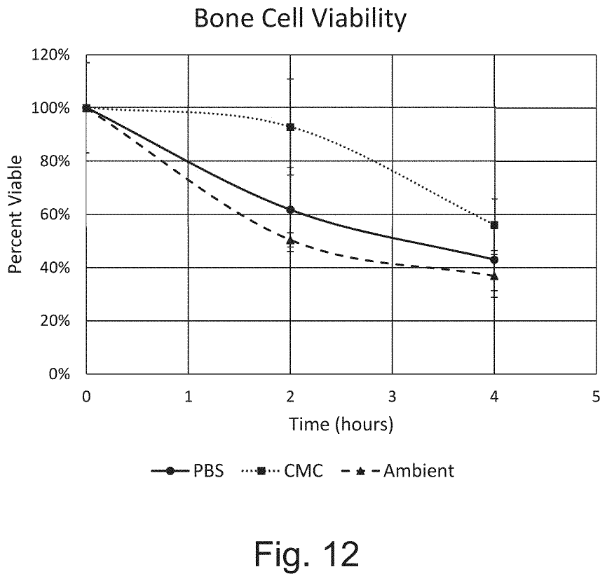

The results of an experiment using a carboxymethyl cellulose gel are illustrated in FIG. 12. Fluorescent intensity results were normalized relative to the 0 hour condition in each figure. FIG. 12 illustrates the relatively rapid decline in bone cell viability over four hours in the control group that was not immersed in saline or CMC gel. Bone cell viability in saline or CMC gel groups also declined with time, although less drastically relative to the control group. As illustrated in FIG. 12, by hour two, the percent of viable cells had dropped to 50% for the control group and 62% and 93% for the saline and CMC gel groups, respectively. By hour four, the percent of viable cells had dropped to 37% for the control group and 43% and 56% for the saline and CMC gel groups, respectively.

The results of these experiments indicate that bone cell viability drops rapidly when left under dry conditions and that a sterile solution or gel, such as saline or a carboxymethyl cellulose gel, may significantly extend the viability of the cells, which may in turn improve the effectiveness of a fusion and/or other treatments.

### Example 5

Gels were created from alginate, carboxymethyl cellulose, or hydroxypropyl methyl cellulose powders dissolved in saline at various concentrations. The relative handling characteristics of these gels were then qualitatively assessed (Table 3) and the viscosity of these gels was measured (Table 3) using a rheometer (Discovery Hybrid Rheometer 30, TA Instruments). The handling results in Table 3 demonstrate that at low gel concentrations, handling for all gels is very poor. As gel concentration begins to increase, handling characteristics become more favorable. Eventually, gel concentration becomes high enough that handling characteristics begin to decrease as the gel begins to crumble with handling. This trend was seen in all 3 gels tested here, although the gel concentration that resulted in optimal handling differed for each gel. The viscosity results in Table 3 demonstrate that all 3 gels are shear-thinning gels, with viscosity decreasing at higher shear rates. Viscosity for all 3 gels also increased at the higher gel concentration.

TABLE 3

| | Gel Agent | Relative | Viscosity | Viscosity | Viscosity |
|---|---|---|---|---|---|
| Gel | Composition | Handling | (Pa*s) @ | (Pa*s) @ | (Pa*s) @ |
| Agent | (w/v) | Grade | 1 1/s | 10 1/s | 100 1/s |
| Alginate | 2% | −− | | | |
| | 3% | − | | | |
| | 4% | + | 100.9 | 47.8 | 13.0 |
| | 6% | ++ | | | |
| | 8% | +++ | 633.0 | 205.0 | 45.4 |
| | 10% | ++ | | | |
| Carboxy- | 3% | −− | | | |
| methyl | 4% | − | | | |
| Cellulose | 6% | + | 131.8 | 38.2 | 9.4 |
| (CMC) | 10% | ++ | | | |
| | 15% | +++ | 1900.0 | 382.5 | 70.0 |
| | 20% | ++ | | | |
| Hydroxy- | 2% | −− | | | |
| propyl | 3% | − | | | |
| methyl | 4% | + | 51.8 | 25.7 | 8.1 |
| cellulose | 6% | ++ | | | |
| (HPMC) | 10% | +++ | 1235.5 | 380.0 | 79.4 |
| | 12% | ++ | | | |

Handling grading scale:
−− = very poor
− = poor
+ = fair
++ = good
+++ = better
Viscosity measurements @ 22° C., shear rate: 1, 10, and 100 recip. sec

Any of the above described gels may be used as the gel composition of Example 4.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of maintaining cellular viability of harvested bone, the method comprising:
(a) providing a source of bone or bone particles;
(b) combining said bone or bone particles with a sterile solution;
(c) storing said bone or bone particles in said sterile solution until introduction into a patient.

2. The method according to claim 1, wherein said providing a source of bone or bone particles comprises harvesting bone from said patient.

3. The method according to claim 1, wherein said sterile solution comprises one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

4. The method according to claim 1, wherein said combining step further comprises wetting said bone or bone particles with said sterile solution.

5. The method according to claim 1, wherein said bone or bone particles comprise cortical bone, cancellous bone, blood, bone marrow, bone marrow aspirate, or any combination thereof.

6. A method of extending cellular viability of harvested bone for an autograft, the method comprising:
(a) providing a source of bone or bone particles;
(b) combining said bone or bone particles with a sterile solution;
(c) storing said bone or bone particles in said sterile solution until autograft introduction into a patient.

7. The method according to claim 6, wherein said providing a source of bone or bone particles comprises harvesting bone from said patient.

8. The method according to claim 6, wherein said sterile solution comprises one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

9. The method according to claim 6, wherein said combining step further comprises wetting said bone or bone particles with said sterile solution.

10. The method according to claim 6, wherein said bone or bone particles comprise cortical bone, cancellous bone, blood, bone marrow, bone marrow aspirate, or any combination thereof.

11. A kit for maintaining cellular viability of harvested bone or bone particles for an autograft, said kit comprising:
(a) a vessel for holding said harvested bone or bone particles; and
(b) a vessel for holding a sterile solution.

12. The kit according to claim 11, wherein said sterile solution comprises a salt solution.

13. The kit according to claim 11, wherein said sterile solution comprises one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

14. The kit according to claim 11, wherein said bone or bone particles comprise cortical bone, cancellous bone, blood, bone marrow, bone marrow aspirate, or any combination thereof.

15. A surgical method for instilling harvested bone or bone particles into a patient comprising:
(a) obtaining a bone or bone particles from the patient;
(b) combining said bone or bone particles with a sterile solution;
(c) maintaining said bone or bone particles in said sterile solution; and
(d) introducing said bone or bone particles into the patient.

16. The method according to claim 15, wherein said combining step further comprises wetting said bone or bone particles with said sterile solution.

17. The method according to claim 15, wherein said sterile solution comprises a salt solution.

18. The method according to claim 15, wherein said sterile solution comprises one or more of saline, a buffer, a preservative, a nutrient, an electrolyte, an excipient, a gelling agent, a carrier, a temperature-sensitive polymer, a solubilizing agent, a stabilizing agent, a tonicity modifier, a bulking agent, a viscosity enhancer or reducer, a surfactant, or any mixture thereof.

19. The method according to claim 15, wherein said bone or bone particles comprise cortical bone, cancellous bone, blood, bone marrow, bone marrow aspirate, or any combination thereof.
